(19) United States
(12) Patent Application Publication
(10) Pub. No.: US 2021/0393650 A1
(43) Pub. Date: Dec. 23, 2021

(54) Designing Treatments for Chemotherapy-Induced and Age-Related Menopausal Symptoms Using Combinations of Natural Products and Prescription Medications

(71) Applicant: Tess Ventures, Inc., Granite Bay, CA (US)

(72) Inventors: Michael W DeGregorio, Granite Bay, CA (US); Gregory T Wurz, Lincoln, CA (US)

(73) Assignee: Tess Ventures, Inc., Granite Bay, CA (US)

(21) Appl. No.: 17/279,552

(22) PCT Filed: Sep. 24, 2019
(86) PCT No.: PCT/US2019/052661

§ 371 Date: Mar. 24, 2021

Related U.S. Application Data
(60) Provisional application No. 62/736,602, filed on Sep. 26, 2018.

(51) Int. Cl. A61K 31/565; A61K 31/55; A61K 31/085; A61K 31/4525; A61K 45/06

(52) U.S. Cl. CPC A61K 31/565; A61K 31/55; A61K 45/06; A61K 31/4525; A61K 31/085

(57) ABSTRACT

The present invention provides methods for alleviating menopausal symptoms as a result of chemotherapy-induced or natural menopause in a subject, the method comprising administering an effective amount of a natural product or combination of natural products tailored to a subjects specific symptoms, with an effective amount of hormonal or non-hormonal synthetic prescription medication. In particular embodiments, the natural product or combination of natural products alleviate vasomotor menopausal symptoms in a subject, while the prescription medication alleviates symptoms associated with genitourinary syndrome of menopause. In some embodiments, the natural product or combination of natural products is prepared in formulations designed for administration at different times of day.

### CROSS-REFERENCES TO RELATED APPLICATIONS

This application claims priority to U.S. Provisional Patent Application No. 62/736,602, filed Sep. 26, 2018, the disclosure of which is hereby incorporated by reference in its entirety for all purposes.

### BACKGROUND OF THE INVENTION

Menopausal symptom treatment history dates back to the late 19th century, when one of the first products, Ovarin, was introduced in the 1890s. Ovarin was marketed until products derived from the urine of pregnant women were developed in the early 1930s, but due to the limited quantities of estrogens in human urine and the high cost associated with their recovery [Davis S R. et al., J Endocrinol 185:207-222 (2005)], other natural sources were sought. This led to the discovery by Bernhard Zondek that the urine of pregnant mares contained large quantities of estrogens [Davis S R. et al., J Endocrinol 185:207-222 (2005)], and it is from this natural source that products such as Premarin® were derived in the late 1930s [Stefanick M L., Am J Med 118:64-73 (2005); Davis S R. et al., J Endocrinol 185:207-222 (2005)]. Premarin® (conjugated equine estrogens; CEE) was approved by the U.S. Food and Drug Administration (FDA) shortly thereafter in 1942, and it remains to this day the leading hormone-based therapy. While the FDA has approved synthetic forms of estrogen for the treatment of menopausal symptoms, Premarin® remains the most commonly prescribed form of estrogen for use as hormone replacement therapy (HRT) [DeGregorio M W. et al., Painful Sex Associated with Menopause: Interpreting FDA Warnings when Choosing a Treatment for Dyspareunia, Hauppauge, N.Y.: Nova Science Publishers (2014)].

The persistent dominance of Premarin® among hormone-based products for the treatment of menopausal symptoms, including vasomotor symptoms (i.e., hot flashes and night sweats) and vulvar and vaginal atrophy (VVA, which includes symptoms of vaginal dryness and dyspareunia), is no accident. To gain FDA approval prior to 1962, new drugs were merely required to be proven safe, rather than efficacious [Stefanick M L., Am J Med 118:64-73 (2005)]. Therefore, Premarin® was never subjected to today's more rigorous FDA approval standards, which in addition to efficacy require that the active ingredient(s) be clearly defined and characterized. To this day, 77 years after its approval, the active components of Premarin® have still not been completely defined [Bhavnani B R. et al., J Steroid Biochem Mol Biol 142:16-29 (2014)]. While 11 different active estrogen compounds have been identified, Premarin® contains a plethora of unidentified molecules that are thought to include more estrogens, progestins and even androgens, none of which have been tested regarding biologic activity and potential side effects in humans. In total, Premarin® may contain upwards of 230 different molecules [Bhavnani B R. et al., J Steroid Biochem Mol Biol 142:16-29 (2014)], which has made it extremely difficult if not impossible to prove bioequivalence, a requirement for generic drugs. In 1997, the FDA's Center for Drug Evaluation and Research (CDER) announced that there would be no future approvals of generic forms of Premarin®, and prior approvals of existing generics were revoked due to concerns over lack of bioequivalence, safety and efficacy under a proposal endorsed by the FDA's Generic Drugs Advisory Committee in 1991 [Stefanick M L., Am J Med 118:64-73 (2005)]. Thus, ironically, as a consequence of strict FDA regulations to which it was never subjected, Premarin® essentially has a monopoly on the market for menopausal symptom treatment with CEE.

Following the approval of Premarin® in 1942, the use of estrogen-based products for the treatment of menopausal symptoms steadily increased until reports of an increased risk of endometrial cancer emerged in 1975 [Smith D C. et al., N Engl J Med 293:1164-1167 (1975); Ziel H K. et al., N Engl J Med 293:1167-1170 (1975)], which consequently led to a dramatic reduction. This trend was reversed once the endometrial protective effects of progestin became established, resulting in a resurgence of the use of estrogen-based hormone therapy for menopausal symptoms through 2001 [Stefanick M L., Am J Med 118:64-73 (2005)]. The following year, results of the Women's Health Initiative (WHI) trial of estrogen plus progestin in postmenopausal women with a uterus showed that HRT was associated with increased risks of breast cancer and coronary heart disease, which led to early termination of this trial [Rossouw J E. et al., JAMA 288:321-333 (2002)]. As a result of these findings as well as the WHI's trial of estrogen alone in hysterectomized postmenopausal women [Anderson G L. et al., JAMA 291:1701-1712 (2004)], the use of HRT declined precipitously, stimulating interest in development of new and safer non-hormonal compounds like ospemifene (see below) for the treatment of menopausal symptoms.

The term “induced menopause,” as distinct from natural menopause, has been officially recognized by the North American Menopause Society (NAMS) and is used to describe menopause that occurs abruptly as a result of surgery, e.g., bilateral oophorectomy, chemotherapy and/or radiotherapy. While many women experience vasomotor symptoms (i.e., hot flashes) as well as symptoms of genitourinary syndrome of menopause (GSM), which include vulvar and vaginal atrophy (VVA), vaginal dryness, itching, painful sexual intercourse (dyspareunia), and urinary symptoms such as urgency, incontinence, painful urination and recurrent urinary tract infections, as they normally progress through the menopausal transition, these symptoms can become more frequent and bothersome among cancer survivors, breast cancer survivors in particular, due to the effects of chemotherapy [Arora N K. et al., Cancer 92:1288-1298 (2001); Burwell S R. et al., J Clin Oncol 24:2815-2821 (2006); Knobf M T., Oncologist 11:96-110 (2006); Mar Fan H G. et al., Ann Oncol 21:983-987 (2010)] and adjuvant hormonal treatments such as aromatase inhibitors [Baumgart J. et al., Menopause 20:162-168 (2013); Cella D. et al., Breast Cancer Res Treat 100:273-284 (2006); Kwan K W. et al., Clin Breast Cancer 9:219-224 (2009); Whelan T J. et al., J Clin Oncol 23:6931-6940 (2005); Whelan T J. et al., Clin Cancer Res 12:1056s-1060s (2006)]. Among younger, premenopausal women being treated for breast cancer, the harsh effects of chemotherapy and surgical removal of the ovaries (oophorectomy) result in the early onset of menopause, and the associated vasomotor and GSM symptoms can be particularly distressing for these patients [Knobf M T., Oncologist 11:96-110 (2006); Rosenberg S M. et al., J Thorac Dis 5:S55-S61 (2013); Stuursma A. et al., Maturitas 111:69-76 (2018); Torino F. et al., Endocr Relat Cancer 19:R21-R33 (2012)]. Approximately 19% of the roughly 269,000 newly diagnosed cases of invasive breast cancer in the United States in 2019 were projected to occur in women under 50 years of age [American Cancer Society, Cancer Facts and Figures 2019, Atlanta, Ga.: American Cancer Society (2019); American Cancer Society, Breast Cancer Facts and Figures 2017-2018, Atlanta, Ga.: American Cancer Society (2018)].

The stress and worry over having breast or other types of cancer coupled with sexual problems as a result of GSM and vasomotor symptoms can lead to feelings of sexual inadequacy, loss of interest in sex, and depression in cancer patients, which can in turn exacerbate their menopausal symptoms and significantly impact their overall quality of life [Burwell S R. et al., J Clin Oncol 24:2815-2821 (2006); Knobf M T., Oncologist 11:96-110 (2006); Ganz P A. et al., Breast Cancer Res Treat 38:183-199 (1996); Ganz P A. et al., J Clin Oncol 17:2371-2380 (1999); Pinto A C. et al., Maturitas 70:343-348 (2011)]. Addressing these sexual problems in breast cancer patients and long-term survivors has become a topic of concern [Green L M., Oncology Nursing News May 7:22-24 (2014); Soldera S V. et al., Breast Cancer Res Treat 172:159-166 (2018)] as the number of women surviving breast cancer continues to increase. According to the American Cancer Society, there were estimated to be approximately 15.5 million cancer survivors in the United States as of early 2016 [American Cancer Society, Cancer Facts and Figures 2018, Atlanta, Ga.: American Cancer Society (2018)], about 3.5 million of which were women with a history of breast cancer [American Cancer Society, Breast Cancer Facts and Figures 2017-2018, Atlanta, Ga.: American Cancer Society (2018)].

Chemotherapy for breast cancer can lead to either temporary or permanent ovarian failure depending on the age of the patient, ovarian reserve at diagnosis, and the type of chemotherapy, with younger patients having a greater chance of recovering ovarian function [Torino F. et al., Endocr Relat Cancer 19:R21-R33 (2012); Goodwin P J. et al., J Clin Oncol 17:2365-2370 (1999); Torino F. et al., Crit Rev Oncol Hematol 89:27-42 (2014)]. For example, female breast cancer patients at least 40 years of age who are treated with the chemotherapy regimen containing cyclophosphamide, melphalan and 5-fluorouracil have a greater than 80% risk of permanent ovarian failure [Torino F. et al., Endocr Relat Cancer 19:R21-R33 (2012)]; however, even younger breast cancer patients who experience a resumption of normal ovarian function will experience an earlier than expected menopause due to the toxic effects of chemotherapy on the ovaries [Knobf M T., Oncologist 11:96-110 (2006); Meirow D., Mol Cell Endocrinol 169:123-131 (2000)]. The resulting sudden loss of estrogen brings about abrupt symptoms of menopause, which can be particularly distressing for younger patients [Rosenberg S M. et al., J Thorac Dis 5:S55-S61 (2013)]. In a study of 824 breast cancer survivors, dyspareunia was significantly increased in those patients who had received adjuvant chemotherapy (N=465) compared to other breast cancer survivors who had never been treated with chemotherapy (N=338). No meaningful differences in vasomotor symptoms were observed in this study [Marino J L. et al., Menopause 23:1000-1008 (2016)]. In a retrospective study evaluating the incidence of chemotherapy-induced menopause in 345 young breast cancer patients (median age 42 years), approximately 13% became menopausal following treatment, which was defined as amenorrhea lasting ≥2 years [Dohou J. et al., Oncology 92:255-263 (2017)].

Breast cancer patients taking aromatase inhibitors such as anastrozole and letrozole, which inhibit almost all estrogen production in the body, resulting in extremely low levels of circulating estrogen, experience menopausal symptoms to a greater degree compared to breast cancer patients not using these therapies [Baumgart J. et al., Menopause 20:162-168 (2013); Kwan K W. et al., Clin Breast Cancer 9:219-224 (2009)]. Women taking tamoxifen can also experience increased symptoms of VVA, particularly dyspareunia [Baumgart J. et al., Menopause 20:162-168 (2013)]. Because of the breast cancer risks associated with estrogen-based menopausal therapy, treatment options for women with hormone-responsive cancers are somewhat limited. Breast cancer patients that fail to get adequate symptom relief from non-hormonal moisturizers and lubricants can discuss the use of short-course, low-dose vaginal estrogen with their doctors [NAMS Advisory Panel, Menopause 24:728-753 (2017)]. For women with non-hormone-responsive breast and gynecologic cancers, their GSM symptoms can be managed with vaginal estrogen therapy [Del Carmen M G., et al., Gynecol Oncol 146:427-435 (2017); Faubion S S. et al., Menopause 25:596-608 (2018)], although this approach is favored by only about 30% of oncologists [Biglia N. et al., Clin Breast Cancer 17:611-617 (2017)].

Chemotherapy for cancers other than breast cancer in premenopausal women also frequently results in induced or iatrogenic menopause, with alkylating agents imparting the greatest risk of ovarian dysfunction in breast and other cancers [Overbeek A. et al., Cancer Treat Rev 53:10-24 (2017)]. In a longitudinal study of lung cancer conducted by the Mayo Clinic Epidemiology and Genetics of Lung Cancer Research Program (Rochester, Minn.), treatment including platinum-based therapies with or without taxanes resulted in a self-report of menopause within a year of diagnosis in a majority (64%) of the 85 women who received chemotherapy in a cohort of 182 premenopausal women. Only 15% of the 94 women in this cohort who did not receive chemotherapy self-reported menopause [Cathcart-Rake E J. et al., Menopause 26:306-310 (2019)]. In a retrospective analysis of premenopausal women with colorectal cancer under the age of 40, approximately 94% of the patients with rectal cancer treated with chemoradiotherapy and adjuvant chemotherapy experienced long-term (>12 months) amenorrhea [Wan J. et al., Clin Colorectal Cancer 14:31-34 (2015)]. Treatment for gynecological cancers, which include ovarian, uterine, cervical, vulvar and vaginal cancer, frequently results in induced menopause, the symptoms of which can be more severe than natural menopause [Hinds L. et al., Menopause Int 16:89-93 (2010); Whicker M. et al., Am J Obstet Gynecol 217:395-403 (2017)]. In a prospective study evaluating menopausal symptoms before and after treatment in a cohort of 124 young women (average age 27 years) with cancer requiring chemotherapy, a significantly greater prevalence of symptoms was observed after treatment compared to pretreatment, which was associated with a decrease in anti-Müllerian hormone. Interestingly, the patients in this cohort experienced more menopausal symptoms before treatment compared to a control group. The risk of menopausal symptoms was not significantly affected by the use of systemic hormone therapy in this study [Cameron K E. et al., Oncology 94:200-206 (2018)]. Due to conflicting and inconclusive clinical data regarding the risk of disease recurrence in women treated for gynecologic cancers [Biglia N. et al., Maturitas 82:296-298 (2015)], the use of estrogen-based hormone replacement therapy in this population of cancer survivors remains controversial [Angioli R. et al., Crit Rev Oncol Hematol 124:51-60 (2018); Edey K A. et al., Cochrane Database Syst Rev 5:CD008830 (2018)].

Among the natural products used for menopausal symptom relief, a member of the buttercup family (Ranunculaceae) and native to eastern North America, black cohosh (Cimicifuga racemosa; also known as Actaea racemosa) is the most intensely studied herbal remedy for menopausal symptoms [Borrelli F. et al., Pharmacol Res 58:8-14 (2008)]. Rhizome extracts of this plant contain a number of different pharmacologically active compounds, including especially the triterpene glycosides actein, 23-epi-26-deoxyactein, cimiracemoside A, cimiracemoside C and 24-O-acetylhydro-shengmanol-3-O-β-D-xylopyranoside [Disch L. et al., J Pharm Sci 106:3642-3650 (2017); Guo Y. et al., J Ethnopharmacol 209:264-282 (2017)]. Recent clinical data has demonstrated significant, dose-dependent improvement in menopausal symptoms experienced by postmenopausal women receiving black cohosh extract compared to placebo. All measures of the Kupperman Index and Greene Climacteric Scale used in these studies, including hot flashes, sweating and insomnia, in particular, showed significant improvement with black cohosh treatment [Drewe J. et al., Phytomedicine 20:659-666 (2013); Mohammad-Alizadeh-Charandabi S. et al., Chin Med 8:20 (2013); Schellenberg R. et al., Evid Based Complement Alternat Med 2012:260301 (2012)]. In Chinese postmenopausal women, a black cohosh extract (Remifemen®) was found to be equivalent to the synthetic steroid tibolone in the treatment of menopausal symptoms, with a superior side effect profile [Bai W. et al., Maturitas 58:31-41 (2007)]. The mechanism(s) of action responsible for the alleviation of menopausal symptoms by black cohosh remain controversial, but may best be described as having the mixed estrogen agonist/antagonist characteristics of the selective estrogen receptor modulators [Viereck V. et al., Trends Endocrinol Metab 16:214-221 (2005)], although this activity does not appear to be mediated through genomic estrogen receptors [Wuttke W. et al., J Steroid Biochem Mol Biol 139:302-310 (2014)], and as neurotransmitter mimetic [Wuttke W. et al., J Steroid Biochem Mol Biol 139:302-310 (2014)]. With respect to the symptoms of vulvovaginal atrophy, i.e., vaginal dryness and dyspareunia, black cohosh has not been shown to have any clinically meaningful benefits [Geller S E. et al., Menopause 16:1156-1166 (2009); Mazaro-Costa R. et al., J Sex Med 7:3695-3714 (2010); Newton K M. et al., Ann Intern Med 145:869-879 (2006); Reed S D. et al., Menopause 15:51-58 (2008)].

Phytoestrogens, or plant-derived, non-steroidal estrogen-like compounds, principally include compounds such as isoflavones, e.g., genistein and daidzein, derived from soybeans and red clover, lignans, e.g., enterodiol and enterolactone, derived primarily from flaxseed, and coumestans, e.g., coumestrol, found in split peas, pinto beans, lima beans, alfalfa and clover sprouts [Duncan A M. et al., Best Pract Res Clin Endocrinol Metab 17:253-271 (2003); Poluzzi E. et al., Curr Med Chem 21:417-436 (2014)]. Despite extensive clinical evaluation of phytoestrogens in the treatment of menopausal symptoms, no clear benefits have been shown [Poluzzi E. et al., Curr Med Chem 21:417-436 (2014); Borrelli F. et al., Maturitas 66:333-343 (2010)]; however, it is difficult to draw any firm conclusions due to the high degree of heterogeneity and placebo effects observed in the published research.

Other compounds, while not technically classified as phytoestrogens, may also be useful in the management of menopausal symptoms. These compounds include herbal products such as ginseng (Panax ginseng C. A. Meyer; Panax quinquefolium), curcumin, derived from turmeric, and ginkgo (Ginkgo biloba). Ginseng roots contain triterpenoid saponins known as ginsenosides, which are believed to be the active components [Attele A S. et al., Biochem Pharmacol 58:1685-1693 (1999)]. Ginseng has been studied for its effects in menopausal symptom treatment and was found to be superior to placebo with respect to depression and well-being; however, no meaningful benefits were seen for vasomotor symptoms, vaginal dryness or dyspareunia [Wiklund I K. et al., Int J Clin Pharmacol Res 19:89-99 (1999)]. In the ovariectomized rat model of menopause, curcumin demonstrated significant suppression of tail skin temperature, suggesting a potential benefit in treating hot flashes [Park S. et al., Genes Nutr 11:2 (2016)]. Ginkgo biloba contains ginkgolides and bilobalides and has primarily been used for memory enhancement. Ginkgo's effects on menopausal symptoms have been clinically evaluated, but no significant benefits were observed other than improvement of mental flexibility [Elsabagh S. et al., J Psychopharmacol 19:173-181 (2005); Hartley D E. et al., Pharmacol Biochem Behav 75:711-720 (2003)].

Cannabis (Cannabis sativa) has been used by human beings for both medicinal and recreational purposes for thousands of years, dating back to at least ancient Egypt. This highly complex plant contains over 100 cannabinoids, also called phytocannabinoids, as well as hundreds of other chemical compounds such as terpenes and flavonoids [ElSohly M L. et al., Prog Chem Org Nat Prod 103:1-36 (2017)]. The primary psychoactive constituent of cannabis is Δ⁹-tetrahydrocannabinol (THC), which exerts its activity by binding to endogenous endocannabinoid receptors known as CB₁ and CB₂ [Maccarrone M. et al., Trends Pharmacol Sci 36:277-296 (2015)]. Discovered in the 1990s, the endocannabinoids anandamide {N-arachidonoylethan-olamine; AEA [Devane W A. et al., Science 258:1946-1949 (1992)]} and 2-arachidonoylglycerol {2-AG; [Mechoulam R. et al., Biochem Pharmacol 50:83-90 (1995); Sugiura T. et al., Biochem Biophys Res Commun 215:89-97 (1995)]}, derived from arachidonic acid, are the two best characterized natural ligands for these receptors. The endocannabinoids, their receptors and associated metabolic enzymes comprise what is known and the endocannabinoid system [Maccarrone M. et al., Trends Pharmacol Sci 36:277-296 (2015)]. Other common cannabinoids found in cannabis include, but are not limited to, cannabidiol (CBD), cannabigerol (CBG), cannabinol (CBN), cannabichromene (CBC), and Δ⁹-tetrahydrocannabivarin (Δ⁹-THCV). Within the cannabis plant, CBD, CBG, THCV and Δ⁹-THC exist largely in their carboxylic acid forms, which undergo decarboxylation when sufficiently heated. In order to become psychoactive, Δ⁹-THC acid requires decarboxylation [Grotenhermen F., Clin Pharmacokinet 42:327-360 (2003)]. In the United States, cannabis has been approved for medicinal use in a total of 34 states as of mid-2019, with recreational use now legal in a total of 11 states and the District of Columbia.

The use of cannabis for the alleviation of menopausal symptoms dates back to at least the late 19th century [Russo E., Journal of Cannabis Therapeutics 2:5-35 (2002)]. Today, treatment benefits for symptoms such as hot flashes, night sweats and insomnia have been ascribed to a number of different cannabis strains that are now widely available in the various U.S. states where medicinal and recreational cannabis has been legalized. While the clinical evidence to support these claims is sparse, there is some scientific basis for the utility of cannabis in treating some menopausal symptoms. There is some evidence supporting interactions between the endocannabinoid system and gonadal hormones [Gorzalka B B. et al., Endocrinology 153:1016-1024 (2012.

Among the pharmacodynamics of cannabis relating to menopausal symptoms, researchers have examined cannabis and cannabinoids with respect to their effects on anxiety, hypothermia and insomnia. In the ovariectomized rat model of menopause, Cannabis sativa seed was found to prevent ovariectomy-induced weight gain, dyslipidemia, depression and anxiety [Saberivand A. et al., Methods Find Exp Clin Pharmacol 32:467-473 (2010)]. In humans, low to moderate doses of Δ⁹-THC and CBD have been shown to exert anxiolytic effects [Tamboro S. et al., Recent Pat CNS Drug Discov 7:25-40 (2012)]. Δ⁹-Tetrahydrocannabinol has a well-characterized, centrally-mediated hypothermic effect [Freeman A S. et al., Life Sci 32:1081-1089 (1983)] that has been used as part of a screening battery for assessing cannabinoid activity [Wiley J L. et al., Eur J Pharmacol 471:185-193 (2003)]. This “cooling” effect has also been observed in rhesus macaques [Taffe M A., Neuroscience 201:125-133 (2012)], although there is conflicting evidence in humans [Dumont G J. et al., J Psychopharmacol 25:478-489 (2011)]. While the hypothermic effects of Δ⁹-THC are mediated through the CB₁ receptor, the mechanism appears to differ from that of the endocannabinoids [Nass S R. et al., J Neuroimmune Pharmacol 10:364-370 (2015)]. With respect to insomnia, Δ⁹-THC and synthetic analogs have been shown to enhance short-term sleep quality, while medium to high doses of CBD have a sedative effect and may reduce symptoms of insomnia [Babson K A. et al., Curr Psychiatry Rep 19:23 (2017)]. In regard to the symptoms of VVA, cannabinoids have not been shown to be of any benefit.

Among non-hormonal prescription medications for the treatment of menopausal symptoms, ospemifene (Osphena®) is a new non-steroidal estrogen receptor agonist/antagonist, also known as a selective estrogen receptor modulator (SERM), from the triphenylethylene chemical class that includes tamoxifen and toremifene, both of which are FDA-approved for the treatment of breast cancer. These compounds are commonly referred to as SERMs because of their unique profiles of mixed estrogen agonist/antagonist effects. Ospemifene was approved by the FDA in February 2013 for the treatment of moderate to severe dyspareunia associated with VVA due to menopause [DeGregorio M W. et al., Steroids 90:82-93 (2014)]. In 2015, the European Medicines Agency (EMA) approved ospemifene under the trade name Senshio® for use in the treatment of moderate to severe symptoms of VVA, including dyspareunia and vaginal dryness, in postmenopausal women who are not candidates for local estrogen therapy. This would include women with a history of breast cancer who have completed therapy and other women following successful treatment for hormone-sensitive malignancies [Nappi R E. et al., Minerva Ginecol 69:370-380 (2017)]. Clinical data suggests that ospemifene is equivalent to or better than local estrogen in the treatment of VVA symptoms [Bruyniks N et al., Climacteric 20:195-204 (2017)]. Ospemifene is also a viable alternative for women who are unwilling or unable to use local estrogen therapy. The term genitourinary syndrome of menopause (GSM) was adopted in 2014 and is now preferred over older terms such as VVA [Wurz G T. et al., Clin Interv Aging 9:1939-1950 (2014)]. Up to 60% of postmenopausal women may be affected by GSM [Calleja-Agius J. et al., Climacteric 12:279-285 (2009); Nappi R E. et al., Cimacteric 17:3-9 (2014); Santoro N. et al., J Sex Med 6:2133-2142 (2009)], the symptoms of which are chronic and progressive without treatment in most postmenopausal women, eventually leading to the deterioration of urogenital health and sexual dysfunction [Mac Bride M B. et al., Mayo Clin Proc 85:87-94 (2010)].

While ospemifene is not currently indicated for use in women with a history of breast cancer in the US, it could prove to be a valuable treatment option for cancer survivors suffering from symptoms of GSM who are unable to use estrogen-based therapies and for women whose symptoms are being exacerbated by the use of aromatase inhibitors. Prior to the approval of ospemifene, existing treatments consisted of moisturizers and lubricants, which do not help the underlying condition [Jenkins M R. et al., Cleve Clin J Med 75:S17-S24 (2008)], or contained estrogen, which when taken systemically in the form of HRT has been associated with an increased risk of breast cancer [Chlebowski R T. et al., JAMA 304:1684-1692 (2010)] and other potentially serious complications [Rossouw J E. et al., JAMA 288:321-333 (2002); Nelson H D. et al., Ann Intern Med 157:104-113 (2012); Rozenberg S. et al., Nat Rev Endocrinol 9:216-227 (2013)].

Following the recent successful completion of a Phase III clinical trial of ospemifene for the treatment of vaginal dryness in postmenopausal women, a supplemental new drug application (sNDA) for this new indication was filed with the FDA in March of 2018, which was subsequently approved in January 2019.

Due to the risks associated with hormone-based therapies for the treatment of menopausal symptoms in women who are naturally menopausal as well as those women who have become menopausal as a result of cancer chemotherapy for breast, gynecologic or other cancers, there is a need in the art for treatment alternatives to hormone-based therapies that provide the benefits of such therapies while also being less toxic. The present invention addresses this need, while providing other advantages as well.

### BRIEF SUMMARY OF THE INVENTION

The present invention provides a method for alleviating menopausal symptoms, either chemotherapy-induced or natural, in a subject, the method comprising administering a hormonal or non-hormonal combination therapy comprised of an effective amount of a natural product or products, or pharmaceutically acceptable salts thereof, and an effective amount of a synthetic prescription therapeutic agent to the subject.

In particular aspects, the present invention provides a method for alleviating chemotherapy-induced menopausal symptoms in a subject, the method including administering an effective amount of the non-hormonal prescription therapeutic agent ospemifene, according to Formula I, bazedoxifene/conjugated estrogens (BZA/CE) according to Formula II, or paroxetine according to Formula III:

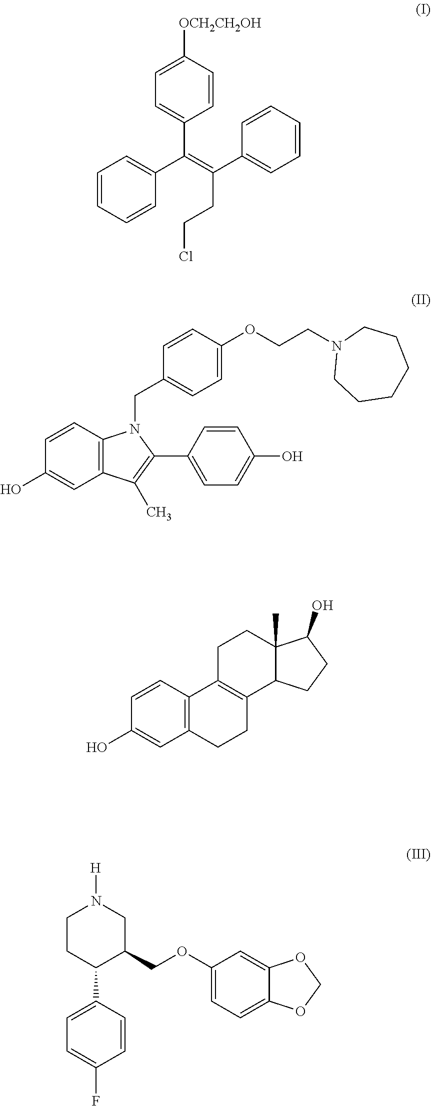

in combination with an effective amount of a natural product or mixture of natural products, or pharmaceutically acceptable salts thereof, to the subject. Formula II shows the chemical structure of bazedoxifene (left) and a non-limiting example (Δ⁸,17β-estradiol; right) of the many steroidal compounds found in conjugated estrogens.

In some embodiments, the subject is experiencing menopausal symptoms as a result of chemotherapy-induced menopause following treatment for breast, gynecological, or other cancers, subjects in whom hormone-based treatment may be contraindicated due to the hormone-responsive nature of their disease.

In particular embodiments, the natural product or natural product mixture for a subject experiencing chemotherapy-induced menopausal symptoms is selected from a group consisting of black cohosh, phytoestrogens, herbal products, cannabinoids, terpenes, and combinations thereof. In some instances, the phytoestrogen is selected from a group consisting of isoflavones, lignans, and coumestans. In other instances, the herbal product is selected from a group consisting of ginseng, curcumin, and Ginkgo biloba.

In some embodiments, the subject is experiencing menopausal symptoms as a result of natural menopause.

In particular embodiments, the natural product or natural product mixture for a subject experiencing menopausal symptoms as a result of natural menopause is selected from a group consisting of black cohosh, phytoestrogens, herbal products, cannabinoids, terpenes and combinations thereof. In some instances, the phytoestrogen is selected from a group consisting of isoflavones, lignans, and coumestans. In other instances, the herbal product is selected from a group consisting of ginseng, curcumin, and Ginkgo biloba.

In some embodiments, the hormonal or non-hormonal synthetic prescription medication (e.g., ospemifene; Formula I) alleviates those menopausal symptoms for which the medication is indicated at the prescribed dose (e.g., dyspareunia and vaginal dryness in the case of ospemifene), while the natural product or products treat those symptoms associated with menopause for which the prescription medication is not indicated (e.g., hot flashes and night sweats in the case of ospemifene). In some instances, the hormonal or non-hormonal synthetic prescription medication (e.g., ospemifene; Formula I) alleviates the menopausal symptoms of dyspareunia and vaginal dryness, while the natural product or products treat other symptoms of menopause (e.g., hot flashes and night sweats). In other instances, the hormonal or non-hormonal synthetic prescription medication [e.g., BZA/CE (Formula II), or paroxetine (Formula III)] alleviates menopausal vasomotor symptoms (e.g., hot flashes and night sweats), while the natural product or products treat other symptoms of menopause (e.g., vaginal dryness and dyspareunia).

In some embodiments, the hormonal or non-hormonal synthetic prescription medication is administered in combination with one or more natural products.

In some embodiments, the effective amount of natural product or combination of natural products is an amount that, when used together with a synthetic hormonal or non-hormonal medication prescribed by a physician, results in the satisfactory alleviation of a subject's specific symptoms. That is, the amount of natural product used as a single agent or as a combination of natural products is individually tailored to the treatment of menopausal symptoms in the subject.

In some embodiments, multiple dose levels of each natural product and various combinations are formulated to allow subjects to tailor their individual treatments to fit their specific symptoms. The phytoestrogen component may consist of an isoflavone (e.g., genestein or daidzein), a lignan (e.g., enterodiol or enterolactone), a coumestan (e.g., coumestrol), curcumin, ginseng, Ginkgo, or other natural product with potentially beneficial effects against menopausal symptoms, or any combination of these agents.

In some embodiments, the ratios between the natural product cannabinoid components can be equal (e.g., 1:1 Δ⁹-THC:CBD), or any other ratio designed to achieve a specific effect (e.g., 3:1 Δ⁹-THC:CBD for the alleviation of hot flashes and night sweats, or 3:1 CBD:Δ⁹-THC for symptoms of anxiety.

In some embodiments, formulations of natural products or combinations of natural products can be designed for use at a particular time of day (e.g., morning or evening) so that unwanted effects may be avoided in the subject (e.g., a 3:1 Δ⁹-THC:CBD cannabinoid combination in the evening when the sedative effects of Δ⁹-THC may be desired, or a 3:1 CBD:Δ⁹-THC cannabinoid combination in the morning so the subject can remain alert while still receiving the benefit of menopausal symptom relief).

In another aspect, the present invention provides a kit including one or more natural products or combinations of natural products.

In some embodiments, the kit also includes a label with instructions for administering the one or more natural products or combinations of natural products together with the prescribed hormonal or non-hormonal synthetic prescription medication.

In some embodiments, the natural product or combination of natural products is selected from a group consisting of black cohosh, phytoestrogens, herbal products, cannabinoids, terpenes and combinations thereof. In some instances, the phytoestrogen is selected from a group consisting of isoflavones, lignans, and coumestans. In other instances, the herbal product is selected from a group consisting of ginseng, curcumin, and Ginkgo biloba.

Other objects, features, and advantages of the present invention will be apparent to one of skill in the art from the following detailed description and figures.

### BRIEF DESCRIPTION OF THE DRAWINGS

FIG. 1. Chemical structures of ospemifene, BZA/CE, and paroxetine. Systematic (IUPAC) designation for ospemifene: Z-2-[4-(4-chloro-1,2-diphenyl-but-1-enyl)-phenoxy]ethanol. IUPAC designation for bazedoxifene: 1-[[4-[2-(azepan-1-yl)ethoxy]phenyl]methyl]-2-(4-hydroxyphenyl)-3-methylindol-5-ol. IUPAC designation for paroxetine: (3S,4R)-3-(1,3-benzodioxol-5-yloxymethyl)-4-(4-fluorophenyl)piperidine.

FIG. 2. Chemical structures of triterpene glycosides found in black cohosh. IUPAC designation for actein (CAS 18642-44-9): (1′R,2S,4'S,4aR,5'S,5aR,7R,7aR,7bR,8R,10R,11aS, 12aS,12bS,14aR)-4′-Hydroxy-1,1,5′,7a,8,12a-hexamethyl-2-(β-D-xylopyranosyloxy)hexa-deca-hydro-2H-spiro[cyclopropa[1′,8a′]naphtho[2′,1′:4,5]indeno[2,1-b]pyran-10,2′-[3,6]dioxabicyclo [3.1.0]hexan]-7-yl acetate. IUPAC designation for 23-epi-26-deoxyactein (CAS 264624-38-6): (1′R,2S,4aR,5′R,5aR,7R,7aR,7bR,8R,10R,11aS,12aS,12bS,14aR)-1,1,5′,7a,8,12a-hexamethyl-2-(β-D-xylopyranosyloxy)hexadecahydro-2H-spiro[cyclopropa[1′,8a′]naphtho[2′,1′:4,5]indeno [2,1-b]pyran-10,2′-[3,6]-dioxabicyclo[3.1.0]hexan]-7-yl acetate.

FIG. 3. Chemical structures of common cannabinoids. (1) Δ⁹-Tetrahydrocannabinol (Δ⁹-THC; CAS 1972-08-3): 6,6,9-trimethyl-3-pentyl-6a,7,8,10a-tetrahydrobenzo[c]chromen-1-ol. (2) Δ⁹-Tetrahydrocannabinolic acid (Δ⁹-THCA; CAS 23978-85-0): (6aR,10aR)-1-hydroxy-6,6,9-tri-methyl-3-pentyl-6a,7,8,10a-tetrahydro-benzo[c]chromene-2-carboxylic acid. (3) Cannabidiol (CBD; CAS 13956-29-1): 2-[(1R,6R)-3-methyl-6-prop-1-en-2-ylcyclohex-2-en-1-yl]-5-pentyl-benzene-1,3-diol. (4) Cannabidiolic acid (CBDA; CAS 1244-58-2): 2,4-dihydroxy-3-[(1R,6R)-3-methyl-6-prop-1-en-2-ylcyclohex-2-en-1-yl]-6-pentylbenzoic acid. (5) Cannabinol (CBN; CAS 521-35-7): 6,6,9-trimethyl-3-pentylbenzo[c]chromen-1-ol. (6) Cannabigerol (CBG; CAS 25654-31-3): 2-[(2E)-3,7-dimethylocta-2,6-dienyl]-5-pentylbenzene-1,3-diol. (7) Cannabigerolic acid (CBGA; CAS 25555-57-1): 3-[(2E)-3,7-dimethylocta-2,6-dienyl]-2,4-dihydroxy-6-pentylbenzoic acid. (8) Cannabichromene (CBC; CAS 20675-51-8): 2-methyl-2-(4-methyl-pent-3-enyl)-7-pentylchromen-5-ol. (9) Δ⁹-Tetrahydrocannabivarin (Δ⁹-THCV; CAS 31262 37-0): (6aR,10aR)-6,6,9-trimethyl-3-propyl-6a,7,8,10a-tetrahydrobenzo[c]chromen-1-ol.

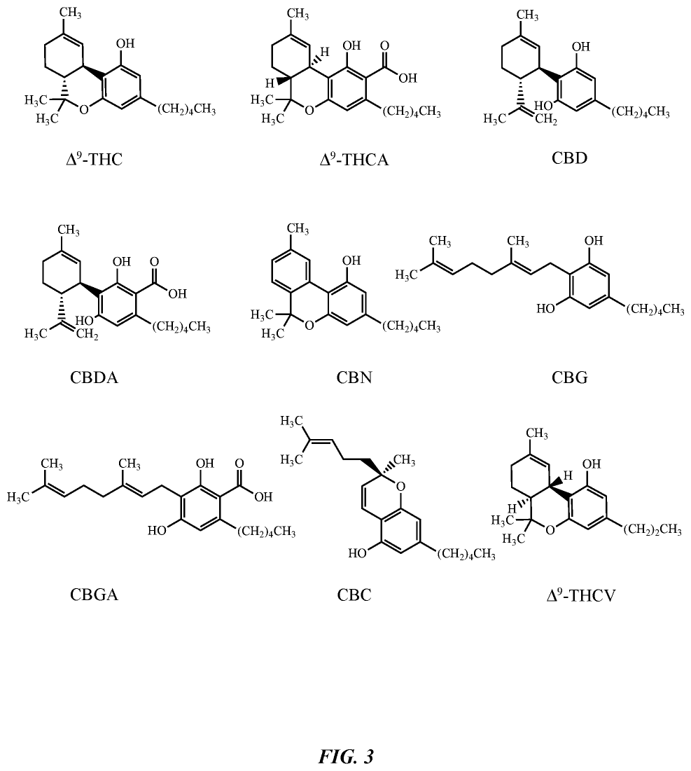

FIG. 4. Chemical structures of common terpenes found in cannabis. (1) α-Bisabolol (CAS 515-69-5): (2R)-6-methyl-2-[(1R)-4-methylcyclohex-3-en-1-yl]hept-5-en-2-ol. (2) trans-Caryophyllene (CAS 87-44-5): (1R,9S,Z)-4,11,11-trimethyl-8-methylenebicyclo[7.2.0]undec-4-ene. (3) p-Cymene (CAS 99-87-6): 1-methyl-4-propan-2-ylbenzene. (4) Guaiol (CAS 489-86 1): 2-[(3S,5R,8S)-3,8-dimethyl-1,2,3,4,5,6,7,8-octahydroazulen-5-yl]propan-2-ol. (5) α-Humulene (CAS 6753-98-6): (1Z,4Z,8Z)-2,6,6,9-tetramethylcycloundeca-1,4,8-triene. (6) Limonene (CAS 138-86-3): 1-methyl-4-prop-1-en-2-ylcyclohexene. (7) Linalool (CAS 78-70-6): 3,7-di-methylocta-1,6-dien-3-ol. (8) Myrcene (CAS 123-35-3): 7-methyl-3-methylideneocta-1,6-diene. (9) cis-Nerolidol (CAS 3790-78-1): (6Z)-3,7,11-trimethyldodeca-1,6,10-trien-3-ol. (10) trans-Nerolidol (CAS 7212-44-4): (6E)-3,7,11-trimethyldodeca-1,6,10-trien-3-ol. (11) α-Pinene (CAS 80-56-8): 4,6,6-trimethylbicyclo[3.1.1]hept-3-ene. (12) β-Pinene (CAS 127-91-3): 6,6-di-methyl-4-methylidenebicyclo[3.1.1]heptane. (13) Terpinolene (CAS 586-62-9): 1-methyl-4-propan-2-ylidenecyclohexene.

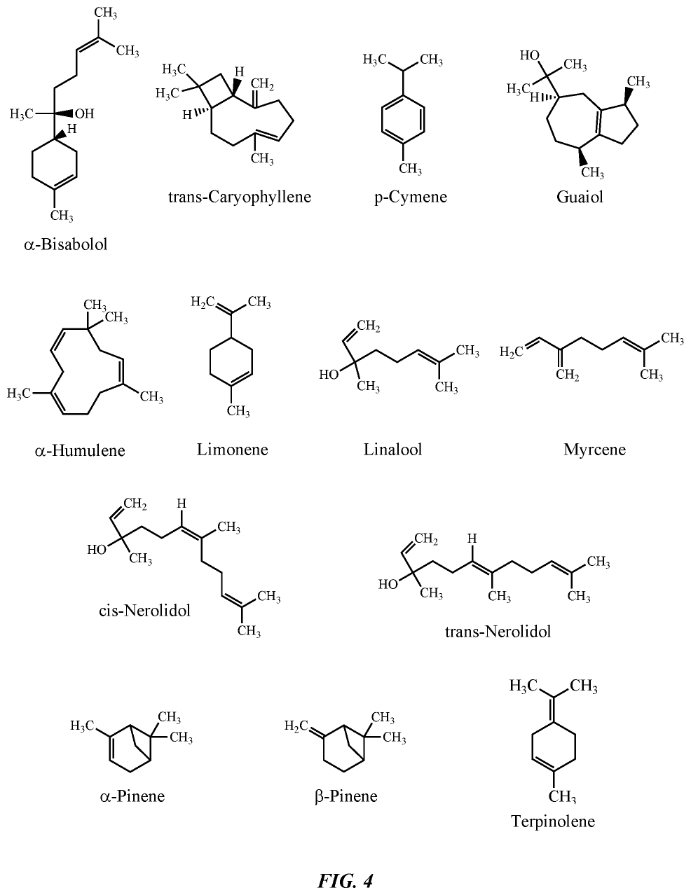

### DETAILED DESCRIPTION OF THE INVENTION

### I. Introduction

This invention involves the development of tailored, hormonal and non-hormonal combinations of natural products, including but not limited to black cohosh, phytoestrogens, cannabinoids, herbal products, and terpenes, with synthetic prescription medications (e.g., ospemifene, BZA/CE, paroxetine) for the alleviation of menopausal symptoms, which include hot flashes, night sweats, insomnia, dyspareunia and vaginal dryness, in perimenopausal women, postmenopausal women who have become menopausal as a result of chemotherapy for breast, gynecologic and other cancers (i.e., chemotherapy-induced menopause), and postmenopausal women who have become menopausal naturally. These novel combinations are designed to provide the benefits of conjugated equine estrogens (CEE) (i.e., biosimilar) without the associated risks of estrogen, the use of which is contraindicated in women with a history of hormone-responsive cancer. The increased risks of breast cancer and coronary heart disease that have been associated with the use of hormone replacement therapy such as Premarin® have become a major concern among healthy postmenopausal women, and in cancer survivors in whom estrogen-based therapies are an option. The treatment combinations developed under this invention will be individually tailored such that cancer survivors suffering from chemotherapy-induced menopausal symptoms, as well as naturally postmenopausal women, would combine a dose of synthetic medication as prescribed by their physicians for a specific indication (e.g., a 60-mg dose for treatment of dyspareunia and/or vaginal dryness associated with GSM in the case of ospemifene), with an amount of black cohosh, phytoestrogens, terpenes, herbal products, or cannabinoids, or combinations thereof, that results in the satisfactory alleviation of their specific symptoms. Treatment combinations may also include other types of prescription medication (e.g., BZA/CE and paroxetine) used at the recommended dosages for treatment of the approved indications (e.g., vasomotor symptoms in the case of BZA/CE and paroxetine).

The dosing regimen will be individually tailored to the subject's specific symptoms by combining the prescription medication (e.g., ospemifene), with a low dose of black cohosh, phytoestrogens, herbal products, terpenes, or cannabinoids, or different combinations of these components. If the subject finds that symptom relief is unsatisfactory, the natural product dosing will be gradually increased until optimum benefits are achieved. Different combinations of natural products and doses may be required to achieve maximum efficacy. These combinations include, but are not limited to, black cohosh with one or more phytoestrogen(s), black cohosh with one more cannabinoids, black cohosh with one or more herbal products, and one or more cannabinoids with one or more phytoestrogens. Natural products may also be used as single agents in combination with a subject's hormonal or non-hormonal synthetic prescription medication. The cannabinoids component may consist of THC (Δ⁸ or Δ⁹), CBD, CBG, CBN, CBC or THCV (Δ⁸ or Δ⁹), or any combination of these, especially THC (Δ⁸ or Δ⁹) and CBD. The ratios between cannabinoid components can be equal (e.g., 1:1 Δ⁹-THC:CBD) or any other ratio designed to achieve a specific effect (e.g., 3:1 Δ⁹-THC:CBD for hot flashes and night sweats, or 3:1 CBD:Δ⁹-THC for symptoms of anxiety). Multiple dose levels of each natural product and various combinations are formulated to allow subjects to tailor their individual treatments to the fit their specific symptoms. The phytoestrogen component may consist of an isoflavone (e.g., genestein or daidzein), a lignan (e.g., enterodiol or enterolactone), or a coumestan (e.g., coumestrol). The herbal product component may consist of curcumin, ginseng (e.g., Panax ginseng C. A. Meyer; Panax quinquefolium, also known as American ginseng), Gingko, or other natural product with potentially beneficial effects against menopausal symptoms, or any combination of these agents. The terpenes component may consist of α-bisabolol, trans-caryophyllene, p-cymene, guaiol, α-humulene, limonene, linalool, myrcene, cis-nerolidol, trans-nerolidol, α-pinene, β-pinene, or terpinolene, or any combination of these.

The natural products and combinations are formulated as tinctures, gels, creams, lotions, patches for transdermal delivery, or other suitable formulation that most appeals to the individual subject. In addition to specific natural products or combinations used to treat a particular menopausal symptom or symptoms, formulations can be designed for use at a particular time of day such as morning or evening so that unwanted effects may be avoided. For example, a 3:1 Δ⁹-THC:CBD cannabinoid combination would be used in the evening when the sedative effects of Δ⁹-THC may be desired, whereas the 3:1 CBD:Δ⁹-THC cannabinoid combination would be used in the morning so that the subject can remain alert while still receiving the benefit of menopausal symptom relief.

### II. Definitions

Unless specifically indicated otherwise, all technical and scientific terms used herein have the same meaning as commonly understood by those of ordinary skill in the art to which this invention belongs. In addition, any method or material similar or equivalent to a method or material described herein can be used in the practice of the present invention. For purposes of the present invention, the following terms are defined.

The terms “a,” “an,” or “the” as used herein not only include aspects with one member, but also include aspects with more than one member. For instance, the singular forms “a,” “an,” and “the” include plural referents unless the context clearly dictates otherwise. Thus, for example, reference to “a natural product” includes a plurality of such natural products, and reference to “a phytoestrogen,” “an herbal product,” “a cannabinoid,” or “a terpene” includes a plurality of such products or compounds known to those skilled in the art, and so forth.

The terms “subject,” “patient,” or “individual” are used herein interchangeably.

As used herein, the term “administering” includes oral administration, topical contact, administration as a suppository, intravenous, intraperitoneal, intramuscular, intralesional, intrathecal, intranasal, or subcutaneous administration to a subject. Administration is by any route, including parenteral and transmucosal (e.g., buccal, sublingual, palatal, gingival, nasal, vaginal, rectal, or transdermal). Parenteral administration includes, e.g., intravenous, intramuscular, intra-arteriole, intradermal, subcutaneous, intraperitoneal, intraventricular, and intracranial. Other modes of delivery include, but are not limited to, the use of liposomal formulations, intravenous infusion, transdermal patches, etc. One skilled in the art will know of additional methods for administering an effective amount of the compounds described herein for the alleviation of menopausal symptoms in an individual.

The term “effective amount” includes an amount or quantity effective, at dosages and for periods of time necessary, to produce a desired (e.g., therapeutic or prophylactic) result with respect to the indicated disease, disorder, or condition. The desired result may comprise a subjective or objective improvement in the recipient of the effective amount. In one non-limiting example, an effective amount of black cohosh extract used in combination with the prescription medication ospemifene includes an amount or dosage sufficient to relieve hot flashes and night sweats. Also, for example, an effective amount of natural product includes an amount or dosage sufficient to decrease symptoms of GSM in a subject taking prescription medication for the treatment of vasomotor symptoms. The effective amount will vary with the type of subject being treated and the compound or combination of compounds applied.

The term “cannabinoid” refers to any member of the broad class of phytocannabinoid compounds derived from the cannabis plant (Cannabis spp.), or their synthetic equivalents, including, but not limited to Δ⁸-THC, Δ⁹-THC, CBD, CBG, CBC, Δ⁸-THCV, Δ⁹-THCV, CBN and any of their associated carboxylic acid forms.

Δ⁸. THC and Δ⁹-THC refer to, respectively, Δ⁸-tetrahydrocannabinol and Δ⁹-tetrahydrocannabinol.

CBD refers to cannabidiol.

CBG refers to cannabigerol.

CBC refers to cannabichromene.

CBN refers to cannabinol.

Δ⁸-THCV and Δ⁹-THCV refer to, respectively, Δ⁸-tetrahydrocannabivarin and Δ⁹-tetrahydrocannabivarin.

The term “natural product” refers to black cohosh, phytoestrogens, terpenes, cannabinoids, and herbal products.

The term “phytoestrogen” refers to any plant-derived natural product that has non-steroidal, estrogen-like activity, including, but not limited to, isoflavones and lignans.

The term “isoflavone” refers to genistein, daidzein, and related compounds.

The term “lignan” refers to enterodiol, enterolactone, and related compounds.

The term “ginseng” refers to Panax ginseng C. A. Meyer, Panax quinquefolium, and related species.

The term “terpene” refers to any of a class of plant-derived monocyclic or bicyclic hydrocarbons, including, but not limited to, α-bisabolol, trans-caryophyllene, p-cymene, guaiol, α-humulene, limonene, linalool, myrcene, cis-nerolidol, trans-nerolidol, α-pinene, β-pinene, terpinolene and related compounds.

The term “herbal product” refers to ginseng, curcumin, Ginkgo biloba, and any other natural product that may have benefits in the management of menopausal symptoms but cannot be classified as a phytoestrogen.

The term “hormonal or non-hormonal prescription medication” refers to ospemifene, bazedoxifene/conjugated estrogens (BZA/CE), paroxetine, or any other non-estrogen therapy prescribed for the treatment of menopausal symptoms in women.

The term “menopausal symptoms” refers to any unpleasant symptoms experienced due to natural or chemotherapy-induced menopause, including, but not limited to, vaginal dryness, dyspareunia, hot flashes, insomnia, and night sweats.

The term “genitourinary syndrome of menopause” or GSM refers to the menopausal symptoms of vulvar and vaginal atrophy (VVA), vaginal dryness, itching, painful sexual intercourse (dyspareunia), and urinary symptoms such as urgency, incontinence, painful urination, and recurrent urinary tract infections.

### III. Description of the Embodiments

A. Hormonal and Non-Hormonal Synthetic Prescription Medications

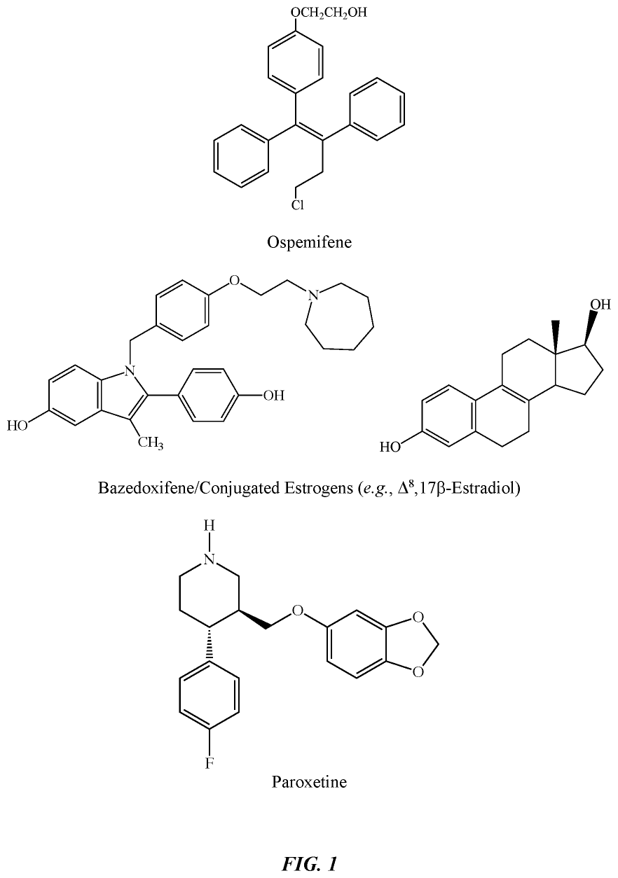

Hormonal and non-hormonal synthetic prescription medications can include any non-estrogen medications that are prescribed for the treatment of menopausal symptoms in post menopausal women. Examples include, but are not limited to, ospemifene (Formula I and FIG. 1), sold under the trade name Osphena® and indicated for the treatment of moderate to severe dyspareunia and vaginal dryness associated with GSM or VVA, BZA/CE (Formula II and FIG. 1), sold under the trade name Duavee® and indicated for the treatment of moderate to severe hot flashes and the prevention of postmenopausal bone loss, and paroxetine (Formula III and FIG. 1), sold under the trade name Brisdelle® and indicated for the treatment of moderate to severe vasomotor symptoms associated with menopause.

B. Natural Products

The natural products of the present invention are used in combination with a subject's hormonal or non-hormonal synthetic prescription medication for the alleviation of menopausal symptoms for which the subject's prescribed medication is not indicated. The natural product or combination of natural products used will depend on the nature of the menopausal symptoms afflicting the subject (e.g., vaginal dryness, dyspareunia, hot flashes, night sweats, insomnia), the prescription medication a subject is using, and whether the subject is menopausal as a result of chemotherapy for the treatment of a hormone-responsive cancer (i.e., chemotherapy-induced) or naturally menopausal.

Non-limiting examples of natural products include black cohosh, phytoestrogens, cannabinoids, herbal products, terpenes, and combinations thereof.

In some embodiments, the natural products of the present invention are agents that possess menopausal symptom alleviating effects that complement those of the subject's prescription medication when administered in combination. As a non-limiting example, a subject experiencing chemotherapy-induced menopausal symptoms being treated with ospemifene for vaginal dryness and/or dyspareunia can be administered a combination of black cohosh, cannabinoids, and terpenes to alleviate the subject's symptoms of hot flashes, night sweats, and insomnia, symptoms that are not addressed by the subject's prescription medication.

1. Black Cohosh

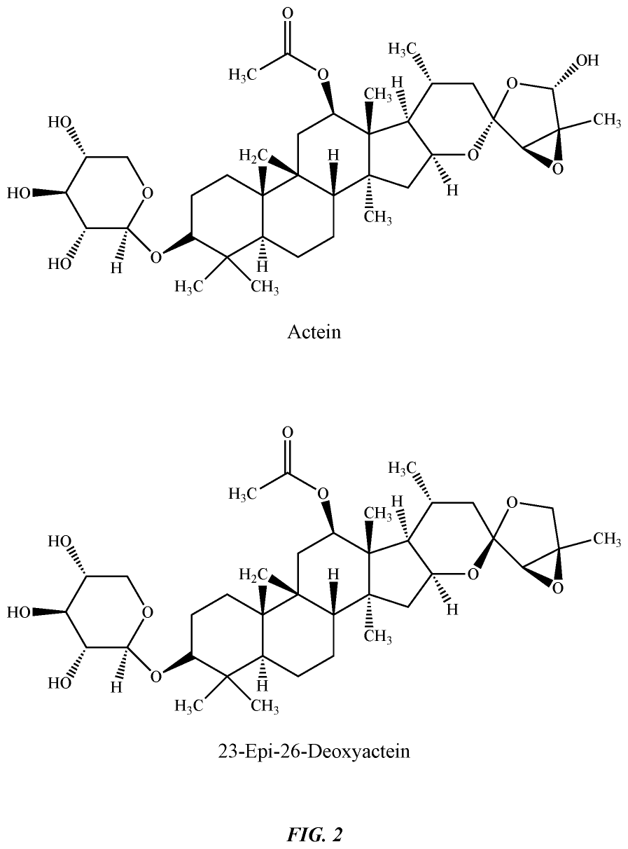

Black cohosh (Cimicifuga racemosa) is an herbal remedy that is well known in the art for the treatment of menopausal symptoms, especially vasomotor symptoms. Extracts of black cohosh contain several pharmacologically active compounds, including triterpene gylcosides (e.g., actein and 23-epi-26-deoxyactein; FIG. 2), that have demonstrated utility in alleviating hot flashes in postmenopausal women.

In particular embodiments, extracts of black cohosh are useful when combined with hormonal and non-hormonal synthetic medications (e.g., ospemifene) that are prescribed for the treatment of menopausal symptoms related to GSM or VVA.

2. Phytoestrogens

Phytoestrogens, which are estrogen-like, non-steroidal compounds derived from plants, include isoflavones from soy beans and red clover, lignans from flaxseed, and coumestans from peas, beans, and clover sprouts. These compounds are well known in the art and have been extensively evaluated clinically for their value in treating menopausal symptoms. Non-limiting examples of phytoestrogens include genistein, daidzein, enterodiol, enterolactone, and coumestrol.

In some embodiments, one or more phytoestrogens are combined with a synthetic prescription medication (e.g., ospemifene) to complement the therapeutic benefits of such medication in the treatment of menopausal symptoms.

3. Cannabinoids

The medicinal qualities of cannabinoids, (e.g., Δ⁹-THC and CBD) are well known in the art, and have been studied for their potential utility in the management of menopausal symptoms. In the treatment of menopausal vasomotor symptoms, cannabinoids such as Δ⁹-THC and CBD have demonstrated some degree of efficacy. Non-limiting examples of cannabinoids include CBD, CBDA, CBG, CBGA, CBC, CBN, Δ⁹-THCV, Δ⁹-THC, and Δ⁹-THCA derived from Cannabis spp. and their synthetic equivalents.

In particular embodiments, a combination of CBD, CBDA, CBG, CBGA, Δ⁹-THC, and Δ⁹-THCA is combined with a synthetic prescription medication (e.g., ospemifene) indicated for the treatment of moderate to severe vaginal dryness and dyspareunia in order to provide additional relief from menopausal vasomotor symptoms in subjects with chemotherapy-induced menopause.

4. Terpenes

Terpenes are ubiquitous among the various plant species that have been studied for their menopausal symptom alleviating effects and are well known in the art. In particular embodiments, the terpene component of cannabis extract, especially including a mixture of the compounds α-bisabolol, trans-caryophyllene, p-cymene, guaiol, α-humulene, limonene, linalool, myrcene, cis-nerolidol, trans-nerolidol, α-pinene, β-pinene, and terpinolene, is combined with a synthetic prescription medication (e.g., ospemifene) to augment and/or complement the efficacy of the prescribed medication in treating menopausal symptoms as a result of chemotherapy-induced menopause.

5. Herbal Products

Other than black cohosh, phytoestrogens, cannabinoids, and terpenes, there are a number of other herbal products that have been studied for their potential efficacy in the treatment of menopausal symptoms. Non-limiting examples include ginseng (e.g., Panax ginseng C. A. Meyer and Panax quinquefolium), curcumin derived from turmeric, and Ginkgo (Ginkgo biloba). These compounds have demonstrated varying degrees of utility in the treatment of menopausal symptoms.

In some embodiments, one or more herbal products may be combined with synthetic prescription medication (e.g., ospemifene) to augment and/or complement the efficacy of the prescribed medication in treating menopausal symptoms.

C. Diseases and Conditions

1. Chemotherapy-Induced Menopause

In certain other aspects, menopausal symptoms occurring as a result of chemotherapy-induced menopause, or simply induced menopause, can be effectively treated using a combination of one or more herbal products and a synthetic prescription medication indicated for such a subject's menopausal symptoms. Induced menopause occurs abruptly as a result of surgery, chemotherapy, and/or radiotherapy for cancer. Compared to women undergoing a natural transition into menopause, menopausal symptoms among cancer survivors can become more frequent and bothersome, particularly among breast cancer survivors, due to the effects of chemotherapy and adjuvant hormonal treatments such as aromatase inhibitors. Menopausal symptoms can be particularly distressing among younger, premenopausal women treated for breast cancer due to the harsh effects of chemotherapy and surgical removal of the ovaries. As the population of long-term cancer survivors continue to grow, so does the need for addressing the menopausal symptom treatment needs in these subjects.

In particular embodiments, a subject experiencing menopausal symptoms as a result of treatment for a hormone-responsive cancer (e.g., breast cancer), is treated with a combination of one or more natural products, especially a mixture of cannabinoids, black cohosh, and terpenes, in combination with a non-hormonal synthetic prescription medication (e.g., ospemifene).

2. Menopausal Symptoms as a Result of Natural Menopause

In certain aspects, menopausal symptoms as a result of natural menopause can be effectively treated by administering one or more natural products in combination with a hormonal or non-hormonal synthetic prescription medication (e.g., ospemifene). Beginning during the menopausal transition (perimenopause) and continuing into postmenopause, women normally experience a number of different symptoms such as hot flashes, night sweats, insomnia, VVA, vaginal dryness, and painful sexual intercourse (dyspareunia), any or all of which can negatively affect their quality of life. Non-limiting examples of the most characteristic and problematic menopausal symptoms include hot flashes and night sweats, which are termed vasomotor symptoms and which are experienced by up to 80% of menopausal women at some point in their lives, insomnia, dyspareunia, and vaginal dryness. These symptoms are a natural result of the declining sex hormone levels that occur in women as they progress through the menopausal transition. Unlike vasomotor symptoms, which usually decrease in frequency and severity with time, symptoms such as vaginal dryness and dyspareunia are usually chronic and progressive without treatment.

In particular embodiments, a subject experiencing menopausal symptoms as a result of natural menopause is treated with a combination of one natural products, especially a mixture of black cohosh, cannabinoids, and terpenes, in combination with a hormonal or non-hormonal synthetic prescription medication (e.g., ospemifene).

D. Pharmaceutical Compositions

The natural products described herein are useful in the manufacture of a pharmaceutical composition or a medicament for alleviating menopausal symptoms in a subject experiencing such symptoms as a result of chemotherapy-induced menopause or natural menopause. In certain aspects, a pharmaceutical composition or medicament comprising one or more natural products can be administered to a subject for the treatment of menopausal symptoms in combination with synthetic hormonal or non-hormonal prescription medication to enhance the efficacy of the prescription medication.

Pharmaceutical compositions or medicaments for use in the present invention can be formulated by standard techniques or methods well-known in the art of pharmacy using one more physiologically acceptable carriers or excipients. Suitable pharmaceutical carriers are described herein and in, e.g., Remington's Pharmaceutical Sciences by E. W. Martin. Compounds and agents of the present invention and their physiologically acceptable salts and solvates can be formulated for administration by any suitable route, including, but not limited to, orally, topically, nasally, rectally, pulmonary, parenterally (e.g., intravenously, subcutaneously, intramuscularly, etc.), and combinations thereof. In some embodiments, the natural products are dissolved in a liquid, for example, water. The most suitable route of administration for a natural product or combination of natural products in any given case will depend, in part, on the type of natural product or products being used as well as the nature and severity of the subject's menopausal symptoms. In embodiments where the natural product or products are administered in combination with a synthetic prescription medication, the natural product or products and prescription medication may be administered by the same or different route of administration. For example, the prescription medication may be administered orally, while the natural product or products may be administered transdermally or topically.

The pharmaceutical compositions or medicaments of the present invention can include one or more natural products or any pharmaceutically acceptable salts thereof, as an active ingredient and a pharmaceutically acceptable carrier and/or excipient or diluent. In some embodiments, the pharmaceutical compositions comprising different natural products are prepared as separate medicaments. In some embodiments, the pharmaceutical compositions comprising different natural products are prepared as a single medicament.

In embodiments where more than one natural product is used, the natural products can be combined as an active ingredient in intimate admixture with a suitable pharmaceutical carrier and/or excipient according to conventional pharmaceutical compounding techniques. Any carrier and/or excipient suitable for the form or preparation desired for administration in contemplated for use with the compounds disclosed herein.

In certain embodiments, the pharmaceutical compositions or medicaments described herein are suitable for systemic administration. Systemic administration includes enteral administration (e.g., absorption of the compound through the gastrointestinal tract) or parenteral administration (e.g., injection, infusion, or implantation). In some embodiments, the pharmaceutical compositions or medicaments may be administered via a syringe or intravenously. In preferred embodiments, the pharmaceutical compositions or medicaments are injected subcutaneously.

In some embodiments, the present invention provides a pharmaceutical composition including black cohosh extract, phytoestrogens, cannabinoids, herbal products, terpenes, and a synthetic prescription medication. In some embodiments, the black cohosh extract, phytoestrogens, cannabinoids, herbal products, terpenes, and synthetic prescription medication are separately prepared pharmaceutical compositions. In some embodiments, the pharmaceutically acceptable excipient includes a salt or a diluent.

In some embodiments, the present invention provides a pharmaceutical composition including a phytoestrogen and a synthetic prescription medication. In some embodiments, the phytoestrogen and the synthetic prescription medication are separately prepared pharmaceutical compositions. In some embodiments, the pharmaceutically acceptable excipient includes a salt or a diluent.

In some embodiments, the present invention provides a pharmaceutical composition including an herbal product and a synthetic prescription medication. In some embodiments, the herbal product and the synthetic prescription medication are separately prepared pharmaceutical compositions. In some embodiments, the pharmaceutically acceptable excipient includes a salt or a diluent.

In some embodiments, the present invention provides a pharmaceutical composition including a combination of one or more natural products and a synthetic prescription medication. In some embodiments, the natural product or combination of natural products and the synthetic prescription medication are separately prepared pharmaceutical compositions. In some embodiments, the pharmaceutically acceptable excipient includes a salt or a diluent.

For oral administration, a pharmaceutical composition or a medicament can take the form of, e.g., a tablet or a capsule prepared by conventional means with a pharmaceutically acceptable excipient. Preferred are tablets and gelatin capsules comprising the active ingredient(s), together with (a) diluents or fillers, e.g., lactose, dextrose, sucrose, mannitol, sorbitol, cellulose (e.g., ethyl cellulose, microcrystalline cellulose), glycine, pectin, polyacrylates and/or calcium hydrogen phosphate, calcium sulfate, (b) lubricants, e.g., silica, anhydrous colloidal silica, talcum, stearic acid, its magnesium or calcium salt (e.g., magnesium stearate or calcium stearate), metallic stearates, colloidal silicon dioxide, hydrogenated vegetable oil, corn starch, sodium benzoate, sodium acetate and/or polyethylene glycol; for tablets also (c) binders, e.g., magnesium aluminum silicate, starch paste, gelatin, tragacanth, methylcellulose, sodium carboxymethylcellulose, polyvinylpyrrolidone and/or hydroxypropyl methylcellulose; if desired, (d) disintegrants, e.g., starches such as potato starch or sodium starch, glycolate, agar, alginic acid or its sodium salt, or effervescent mixtures; (e) wetting agents, e.g., sodium lauryl, sulfate, and/or (f) absorbents, colorants, flavors and sweeteners. In some embodiments, the tablet contains a mixture of hydroxypropyl methyl cellulose, polyethylene glycol 6000 and titanium dioxide. Tablets may be either film coated or enteric coated according to methods known in the art.

Liquid preparations for oral administration can take the form of, e.g., solutions, syrups, suspensions, and tinctures, or they can be presented as a dry product for constitution with water or other suitable vehicle before use. Such liquid preparations can be prepared by conventional means with pharmaceutically acceptable additives, e.g., suspending agents such as sorbitol syrup, cellulose derivatives, or hydrogenated edible fats; emulsifying agents such as lecithin or acacia; non-aqueous vehicles, e.g., almond oil, oily esters, ethyl alcohol, or fractionated vegetable oils; and preservatives, e.g., methyl- or propyl-p-hydroxybenzenes or sorbic acid. The preparations can also contain buffer salts, flavoring, coloring, and/or sweetening agents as appropriate. If desired, preparations for oral administration can be suitably formulated for any type of administration, e.g., intradermal, subdermal, intravenous, intramuscular, or intranasal, with a syringe or other devices. Formulation for administration by inhalation (e.g., aerosol), or for oral, rectal, or vaginal administration is also contemplated.

Pharmaceutical compositions for pulmonary administration include, but are not limited to, dry powder compositions consisting of the powder of a compound described herein, or a salt thereof, and the powder of a suitable carrier and/or lubricant. The compositions for pulmonary administration can be inhaled from any suitable dry powder inhaler device known to a person skilled in the art. In certain instances, the compositions may be conveniently delivered in the form of an aerosol spray from pressurized packs or a nebulizer, with the use of a suitable propellant, e.g., dichlorodifluoromethane, trichlorofluoromethane, dichlorotetrafluoroethane, carbon dioxide, or other suitable gas. In the case of a pressurized aerosol, the dosage unit can be determined by providing a valve to deliver a metered amount. Capsules and cartridges of, e.g., gelatin for use in an inhaler or insufflator can be formulated containing a powder mix of the compound(s) and a suitable powder base such as lactose or starch.

The natural products of the present invention can also be formulated in rectal compositions such as suppositories or retention enemas, e.g., containing conventional suppository bases such as cocoa butter or other glycerides.

The compositions set forth herein can be formulated for parenteral administration by injection, e.g., by bolus injection. Formulations for injection can be presented in unit dosage form, e.g., in ampoules or in multi-dose containers, with an added preservative. Injectable compositions are preferably aqueous isotonic solutions or suspensions, and suppositories are preferably prepared from fatty emulsions or suspensions. The compositions may be sterilized and/or contain adjuvants, such as preserving, stabilizing, wetting or emulsifying agents, solution promoters, salts for regulating the osmotic pressure and/or buffers. Alternatively, the compound(s) can be in powder form for reconstitution with a suitable vehicle such as sterile pyrogen-free water, before use. In addition, they may also contain other therapeutically valuable substances. The compositions are prepared according to conventional mixing, granulating or coating methods, respectively, and contain approximately 0.1 to 75%, preferably about 1 to 50%, of the compound(s).

In some embodiments, the compounds are prepared with polysaccharide such as chitosan or derivatives thereof (e.g., chitosan succinate, chitosan phthalate, etc.), pectin and derivatives thereof (e.g., amidated pectin, calcium pectinate, etc.), chondroitin and derivatives thereof (e.g., chondroitin sulfate), and alginates.

In some embodiments, the compositions further include a pharmaceutical surfactant. In other embodiments, the compositions further include a cryoprotectant. Non-limiting examples of cryoprotectants include glucose, sucrose, trehalose, lactose, sodium glutamate, PVP, cyclodextrin, 2-hydroxypropyl-13-cyclodextrin (HP13CD) glycerol, maltose, mannitol, saccharose, and mixtures thereof.

E. Methods of Administration

Pharmaceutical compositions or medicaments comprising a natural product or products can be administered to a subject at a therapeutically effective dose in combination with an effective amount of synthetic hormonal or non-hormonal prescription medication, taken as prescribed by the subject's physician, to more effectively alleviate the subject's menopausal symptoms, as described herein. In some embodiments, the pharmaceutical composition or medicament comprising a natural product or product is administered to a subject in an amount sufficient in combination with an effective amount of synthetic prescription medication to elicit an effective therapeutic response in the subject. In some embodiments, the pharmaceutical composition or medicament comprising a natural product or products can be administered to a subject at a therapeutically effective dose in combination with an effective amount of a synthetic prescription medication (e.g., ospemifene) to effectively alleviate menopausal vasomotor symptoms as a result of chemotherapy-induced menopause. In some embodiments, the pharmaceutical composition or medicament comprising a natural product or products can be administered in combination with an effective amount of synthetic prescription medication to effectively alleviate vaginal dryness and dyspareunia in a subject experiencing chemotherapy-induced menopausal symptoms.

The combination therapy described herein includes simultaneous administration as well as sequential administration. In some embodiments, the pharmaceutical composition or medicament comprising a natural product or products is administered on a different time schedule than the synthetic prescription medication. As a non-limiting example, the pharmaceutical composition or medicament may be administered weekly, while the synthetic prescription medication (e.g., ospemifene) may be administered daily, or the pharmaceutical composition or medicament may be administered daily, while the synthetic prescription medication is administered weekly. The administration schedule of the prescription medication is dependent on the type of medication and the symptoms for which it is prescribed by the subject's physician.

In some embodiments, one or more natural products are administered to a subject in combination with synthetic prescription medication to alleviate vasomotor symptoms as a result of chemotherapy-induced menopause.

In particular embodiments, one or more natural products are administered to a subject in combination with ospemifene (Formula I) to alleviate vasomotor symptoms as a result of chemotherapy-induced menopause.

In some embodiments, one or more natural products are administered to a subject in combination with synthetic prescription medication (e.g., ospemifene) to alleviate vasomotor symptoms as a result of natural menopause.

In particular embodiments, one or more natural products are administered to a subject in combination with ospemifene (Formula I) to alleviate vasomotor symptoms as a result of natural menopause.

In some embodiments, one or more natural products are administered to a subject in combination with synthetic prescription medication (e.g., paroxetine, Formula III) to alleviate symptoms of vaginal dryness and dyspareunia as a result of chemotherapy-induced menopause.

In some embodiments, one or more natural products are administered to a subject in combination with synthetic prescription medication (e.g., paroxetine, Formula III) to alleviate symptoms of vaginal dryness and dyspareunia as a result of natural menopause.

In some embodiments, one or more natural products are administered to a subject without a synthetic prescription medication to alleviate vasomotor symptoms as a result of chemotherapy-induced menopause.

In some embodiments, one or more natural products are administered to a subject without a synthetic prescription medication to alleviate symptoms of vaginal dryness and dyspareunia as a result of chemotherapy-induced menopause.

In some embodiments, one or more natural products are administered to a subject without a synthetic prescription medication to alleviate vasomotor symptoms as a result of natural menopause.

In some embodiments, one or more natural products are administered to a subject without a synthetic prescription medication to alleviate symptoms of vaginal dryness and dyspareunia as a result of natural menopause.

In some embodiments, the methods of alleviating menopausal symptoms described herein are administered to a subject with chemotherapy-induced or natural menopause. In certain embodiments, the therapeutic agent is a natural product or combination of natural products combined with a hormonal or non-hormonal synthetic prescription medication.

The formulations of the present invention are useful in the manufacture of a pharmaceutical composition or a medicament. A pharmaceutical composition or medicament can be administered to a subject in need thereof, e.g., a subject experiencing menopausal symptoms as a result of chemotherapy-induced or natural menopause.

In certain methods of alleviating menopausal symptoms, set forth herein, the methods comprise administering a natural product or combination of natural products together with a hormonal or non-hormonal synthetic prescription medication. The natural products may be administered simultaneously with the prescription medication, or they may be administered according to a dosing schedule that best fits the subject's symptoms.

F. Dosage

Pharmaceutical compositions or medicaments comprising a natural product or products in combination with a hormonal or non-hormonal synthetic prescription medication can be administered to a subject at a therapeutically effective dose to alleviate menopausal symptoms as a result of chemotherapy-induced or natural menopause as described herein. The pharmaceutical compositions or medicaments are administered to a subject in an amount sufficient to elicit an effective therapeutic response in the subject.

The dosage of compounds administered is dependent on the subject's body weight, age, individual condition, and/or on the form of administration. The size of the dose will also be determined by the existence, nature, and extent of any adverse effects that accompany the administration of a particular compound in a particular subject. Typically, a dosage of the active compounds is a dosage that is sufficient to achieve the desired effect. Optimal doses and dosing schedules of natural product compositions can be determined on an individualized basis based on the needs of a particular subject. In general, dosage may be given once or more daily, weekly, or monthly. Persons of ordinary skill in the art can easily determine optimum dosages, dosing methodologies, and repetition rates.

The effective amount of a hormonal or non-hormonal synthetic prescription medication administered in combination with a natural product or combination of natural products may be provided at the dosages generally recommended for each prescription medication used.

In some embodiments, a unit dosage for oral administration of a natural product or combination of natural products to a subject (e.g., a human) of about 50 to about 70 kg may contain about 1 and about 500 mg, about 5 and about 500 mg, about 5 and about 250 mg, about 25 to about 250 mg, about 100 and about 1000 mg, about 200 and about 2000 mg, about 500 and about 5000 mg, or about 1000 and about 2000 mg of the compound(s). In particular embodiments, a unit dosage for oral administration of natural product or combination of natural products to a subject (e.g., a human) of about 50 to about 70 kg may contain about 5 mg, 10 mg, 15 mg, 20 mg, 25 mg, 30 mg, 35 mg, 40 mg, 45 mg, 50 mg, 75 mg, 100 mg, 200 mg, 300 mg, 400 mg, 500 mg, 600 mg, 700 mg, 800 mg, 900 mg, 1000 mg, 1250 mg, 1500 mg, 2000 mg, 2500 mg, 3000 mg, or more of the compound(s).

In some embodiments, a unit dosage for topical administration of natural product or combination of natural products to a subject (e.g., a human) of about 50 to about 70 kg may contain between 1 and about 100 mg, about 5 and about 100 mg, about 5 and about 50 mg, about 5 and about 25 mg, about 5 and about 10 mg, about 2.5 to about 50 mg, about 2.5 to about 25 mg, about 1 to about 50 mg, about 1 to about 25 mg, or about 1 to about 10 mg of the compound(s). In particular embodiments, a unit dosage for topical administration of a natural product or combination of natural products, to a subject (e.g., a human) of about 50 to about 70 kg may contain about 1 mg, 2 mg, 3 mg, 4 mg, 5 mg, 10 mg, 15 mg, 20 mg, 25 mg, 30 mg, 35 mg, 40 mg, 45 mg, 50 mg, 75 mg, 100 mg, or more of the compound(s).

When or more of the natural product compositions of the invention is administered to a subject, a relatively low dose is recommended at first, with the subject subsequently increasing the dose until an appropriate response is achieved (i.e., satisfactory alleviation of the subject's menopausal symptoms). In addition, it is understood that the specific dose level for any particular subject will depend upon a variety of factors including the activity of the specific composition employed, the age, body weight, general health, and diet of the subject, the time of administration, the route of administration, the rate of excretion, any drug combination, and the nature and degree of severity of the menopausal symptoms to be treated.

In some embodiments, a pharmaceutical composition or medicament of the present invention is administered orally once per day or divided into sub-doses and administered in multiple doses, e.g., twice, three times, or four times per day. In particular embodiments, a pharmaceutical composition comprised of a natural product or combination of natural products is administered twice per day, with or without a synthetic prescription medication (e.g., ospemifene), with each dose formulated specifically for the time of day (e.g., a morning dose and an evening dose). As a non-limiting example, the morning dose is formulated to include a 3:1 CBD:Δ⁹-THC cannabinoid ratio, and the evening dose is formulated to include a 3:1 Δ⁹-THC:CBD cannabinoid ratio. However, as will be appreciated by a skilled artisan, oral compositions described herein may be administered in different amounts and at different times.

In some embodiments, a pharmaceutical composition or medicament of the present invention is administered topically once per day or divided into sub-doses and administered in multiple doses, e.g., twice, three times, or four times per day. In particular embodiments, a pharmaceutical composition comprised of a natural product or combination of natural products is administered twice per day, with or without a synthetic prescription medication (e.g., ospemifene), with each dose formulated specifically for the time of day (e.g., a morning dose and an evening dose). As a non-limiting example, the morning dose is formulated to include a 3:1 CBD:Δ⁹-THC cannabinoid ratio, and the evening dose is formulated to include a 3:1 Δ⁹-THC:CBD cannabinoid ratio. However, as will be appreciated by a skilled artisan, topical compositions described herein may be administered in different amounts and at different times.

In some embodiments, the natural product or products are administered for about 1 to about 31 days, e.g., 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, or 31 days. In some embodiments, the natural product or products are administered for at least 1 day. In other embodiments, the natural product or products are administered for one or more weeks, e.g., 1, 2, 3, 4, 5, 6, 7, 8, 9, 10, 11, 12, 13, 14, or more weeks. In yet other embodiments, the natural product or products are administered for one or more months, e.g., 1, 2, 3, 4, 5, 6, 7, 8, 9, 10, 11, 12, or more months.

To achieve the desired therapeutic effect, the natural product or combination of natural products may be administered in combination with synthetic prescription medication for multiple days, weeks, months, or years at the therapeutically effective daily dose. Thus, therapeutically effective administration of the natural product or natural product combinations to alleviate pertinent menopausal symptoms described herein in a subject requires periodic (e.g., daily or twice daily) administration that continues for a period ranging from a few days, to a few weeks, to few months or longer. While consecutive daily doses are a preferred route to achieve a therapeutically effective dose, a therapeutically beneficial effect can be achieved even if the agents are not administered daily, so long as the administration is repeated frequently enough to maintain a therapeutically effective concentration of the agents in a subject. For example, one can administer the agent every day, every other day, or if higher dose ranges are employed and tolerated by the subject, twice a week.

The dosage of a pharmaceutical composition or medicament of the present invention can be monitored and adjusted throughout treatment, depending on the severity of symptoms, frequency of recurrence, and/or the physiological response to the therapeutic regimen. Those of skill in the art commonly engage in such adjustments in therapeutic regimens. Single or multiple administrations of the pharmaceutical compositions or medicaments can be administered depending on the dosage and frequency as required and tolerated by the subject. In any event, the composition or medicament should provide a sufficient quantity of the compounds of the invention to effectively treat a subject. Generally, the dose is sufficient to alleviate menopausal symptoms without producing unacceptable toxicity or side effects in a subject.

G. Kits, Container, Devices, and Systems

A wide variety of kits and systems can be prepared according to the present invention, depending on the intended user of the kit and system and the particular needs of the user. In some aspects, the present invention provides a kit that includes one or more natural products selected from a phytoestrogen, black cohosh, a cannabinoid, an herbal product, a terpene, and combinations thereof. In certain embodiments, the kit includes black cohosh, cannabinoids, and terpenes.

Some of the kits described herein include a label describing a method of administering one or more natural products to be used in combination with a subject's prescription medication for menopausal symptoms.

The compositions of the present invention, including but not limited to compositions comprising one or more natural products described herein, may, if desired, be presented in a bottle, inhaler, jar, vial, ampoule, tube, or other container closure system, which may provide one or more dosages containing the compounds. In certain aspects, the kit may include a formulation or composition as described herein, a container closure system including the formulation or a dosage unit form including the formulation, and a notice or instructions describing a method of use as described herein.

In some embodiments, the kit includes a container which is compartmentalized for holding the various elements of a formulation (e.g., the dry ingredients and the liquid ingredients) or composition, instructions for making the formulation or composition, and instructions for administering the formulation or composition for alleviating menopausal symptoms in a subject who is experiencing chemotherapy-induced or natural menopause.

In some embodiments, the instructions include timing information on when to administer one or more natural product or natural product combinations. In some embodiments, different formulations of natural products are included for use at particular times of day (e.g., morning or evening). A non-limiting example is a kit that includes instructions for administering formulations containing different ratios of the cannabinoids CBD and Δ⁹-THC. In particular embodiments, the cannabinoid ratios are 3:1 CBD: Δ⁹-THC and 3:1 Δ⁹-THC:CBD.

In certain embodiments, the kit may include the pharmaceutical preparation in dehydrated or dry form, with instructions for its rehydration (or reconstitution) and administration.

Kits with unit doses of the compounds described herein, e.g., in oral, inhalant, topical, or transdermal doses, are provided. In such kits, an information package insert describing the use and attendant benefits of the composition for alleviating menopausal symptoms in a subject experiencing chemotherapy-induced or natural menopause may be included in addition to the containers containing the unit doses.

Some embodiments of the present invention include packages that include one or more natural products or one or more natural product combinations as described herein.

### IV. Examples

The following examples are offered to illustrate, but not to limit, the claimed invention.

In particular, Examples 1 and 2 illustrate the formulation of natural product tinctures for oral administration in accordance with embodiments of the invention.

### Example 1: Formulation of IMT-121 (Morning “AM” Dose)

A tincture for oral administration for the alleviation of menopausal symptoms was formulated use in the morning (“AM Dose”) in combination with the non-hormonal synthetic prescription medication ospemifene. This tincture was formulated to contain cannabinoids, terpenes, and black cohosh extract. The cannabinoids and terpenes were obtained by ethanol extraction of different strains of commercially available cannabis containing varying amounts of CBD and Δ⁹-THC. These extracts are prepared in both activated form, whereby the extract is heated for a sufficient amount of time at sufficient temperature known in the art to convert the carboxylic acid forms of the cannabinoids to their active forms, and in inactivated form, which retains the natural content of the active and inactive forms of the major cannabinoids. The cannabinoid and terpene profiles of the IMT-121 AM Dose tincture are shown in the tables below. Concentrations of cannabinoids and terpenes were determined using validated HPLC and GC-FID analytical methodologies, respectively.

TABLE 1
IMT-121 AM Dose Cannabinoid Profile

| Cannabinoid | Concentration (mg/mL) |
|---|---|
| Cannabidiolic Acid (CBDA) | 3.397 |
| Cannabidiol (CBD) | 3.172 |
| Cannabigerol (CBG) | 0.301 |
| Cannabigerolic Acid (CBGA) | 0.267 |
| Δ⁹-Tetrahydrocannabinol (Δ⁹-THC) | 1.475 |
| Δ⁹-Tetrahydrocannabinolic Acid (Δ⁹-THCA) | 1.312 |
| Total Cannabinoids | 9.924 |

TABLE 2
IMT-121 AM Dose Terpene Profile

| Terpene | Concentration (mg/mL) |
|---|---|
| α-Pinene | 0.031 |
| β-Pinene | 0.014 |
| Myrcene | 0.043 |
| p-Cymene | 0.001 |
| Limonene | 0.012 |
| Terpinolene | 0.004 |
| Linalool | 0.008 |
| trans-Caryophyllene | 0.156 |
| α-Humulene | 0.051 |
| cis-Nerolidol | 0.032 |
| trans-Nerolidol | 0.115 |
| Guaiol | 0.007 |
| α-Bisabolol | 0.020 |
| Total Terpenes* | 0.494 |

*Includes unidentified terpenes.

Black cohosh was obtained from an extract containing 333 mg/mL herb weight equivalence. The triterpene glycoside content profile, determined by LC-MS/MS analytical methodology, is as follows:

TABLE 3
IMT-121 AM Dose Triterpene Glycoside Profile

| Triterpene Glycoside | Concentration (mg/mL) |
|---|---|
| Actein | 22.2 |
| 23-epi-26-deoxyactein | 22.2 |
| Total Triterpene Glycosides | 44.4 |

The IMT-121 AM Dose tincture for oral administration was prepared by combining the following materials in a 30-mL amber glass bottle fitted with glass dropper: (1) 7.5 mL of a 50%/50% (v/v) activated/inactivated extract of a high-CBD cannabis strain; (2) 1.0 mL of black cohosh extract (333 mg/mL); (3) 1.5 mL of 95% ethanol; (4) 5.0 mL of deionized water. The subject being treated for menopausal symptoms will take their prescribed daily dose of ospemifene, as directed by their physician, in combination with the described tincture designed to alleviate those menopausal symptoms for which ospemifene is not indicated (e.g., hot flashes, night sweats, insomnia).

### Example 2: Formulation of IMT-121 (Evening “PM” Dose)

A tincture for oral administration for the alleviation of menopausal symptoms was formulated use in the evening (“PM Dose”) in combination with the non-hormonal synthetic prescription medication ospemifene. This tincture was formulated to contain cannabinoids, terpenes, and black cohosh extract. The cannabinoids and terpenes were obtained by ethanol extraction of different strains of commercially available cannabis containing varying amounts of CBD and Δ⁹-THC. These extracts were prepared in both activated form, whereby the extract was heated for a sufficient amount of time at a sufficient temperature known in the art to convert the carboxylic acid forms of the cannabinoids to their active forms, and in inactivated form, which retains the natural content of the active and inactive forms of the major cannabinoids. The cannabinoid and terpene profiles of the IMT-121 PM Dose tincture are shown in the tables below. Concentrations of cannabinoids and terpenes were determined using validated HPLC and GC-FID analytical methodologies, respectively.

TABLE 4
IMT-121 PM Dose Cannabinoid Profile

| Cannabinoid | Concentration (mg/mL) |
|---|---|
| Cannabidiolic Acid (CBDA) | 3.397 |
| Cannabidiol (CBD) | 3.172 |
| Cannabigerol (CBG) | 0.238 |
| Cannabigerolic Acid (CBGA) | 0.133 |
| Δ⁹-Tetrahydrocannabinol (Δ⁹-THC) | 4.049 |
| Δ⁹-Tetrahydrocannabinolic Acid (Δ⁹-THCA) | 1.312 |
| Total Cannabinoids | 12.301 |

TABLE 5
IMT-121 PM Dose Terpene Profile

| Terpene | Concentration (mg/mL) |
|---|---|
| α-Pinene | 0.031 |
| β-Pinene | 0.014 |
| Myrcene | 0.044 |
| p-Cymene | 0.001 |
| Limonene | 0.013 |
| Terpinolene | 0.004 |
| Linalool | 0.007 |
| trans-Caryophyllene | 0.162 |
| α-Humulene | 0.053 |
| cis-Nerolidol | 0.070 |
| trans-Nerolidol | 0.125 |
| Guaiol | 0.007 |
| α-Bisabolol | 0.046 |
| Total Terpenes | 0.577 |

*Includes unidentified terpenes.

Black cohosh was obtained from an extract containing 333 mg/mL herb weight equivalence. The triterpene glycoside content profile, determined by LC-MS/MS analytical methodology, is as follows:

TABLE 6
IMT-121 PM Dose Triterpene Glycoside Profile

| Triterpene Glycoside | Concentration (mg/mL) |
|---|---|
| Actein | 22.2 |
| 23-epi-26-deoxyactein | 22.2 |
| Total Triterpene Glycosides | 44.4 |

The IMT-121 PM Dose tincture for oral administration was prepared by combining the following materials in a 30-mL amber glass bottle fitted with glass dropper: (1) 7.5 mL of a 50%/50% (v/v) activated/inactivated extract of a high-CBD cannabis strain; (2) 1.0 mL of black cohosh extract (333 mg/mL); (3) 1.5 mL of an activated extract of a high-Δ⁹-THC cannabis strain; (4) 5.0 mL of deionized water. The subject being treated for chemotherapy-induced or natural menopausal symptoms will take their prescribed daily dose of ospemifene, as directed by their physician, in combination with the described tincture designed to alleviate those menopausal symptoms for which ospemifene is not indicated (e.g., hot flashes, night sweats, insomnia).

Although the foregoing invention has been described in some detail by way of illustration and example for purposes of clarity of understanding, one of skill in the art will appreciate that certain changes and modifications may be practiced within the scope of the appended claims. In addition, each reference provided herein is incorporated by reference in its entirety to the same extent as if each reference was individually incorporated by reference.

What is claimed is:

1. A method for alleviating chemotherapy induced or age related menopausal symptoms in a subject, the method comprising administering an effective amount of a natural product or combination of natural products, or pharmaceutically acceptable salts thereof, in combination with an effective amount of a hormonal or non-hormonal prescription medication to the subject.

2. The method of claim 1, wherein the prescription medication is ospemifene, represented by Formula I, bazedoxifene/conjugated estrogens, represented by Formula II, or paroxetine, represented by Formula III,

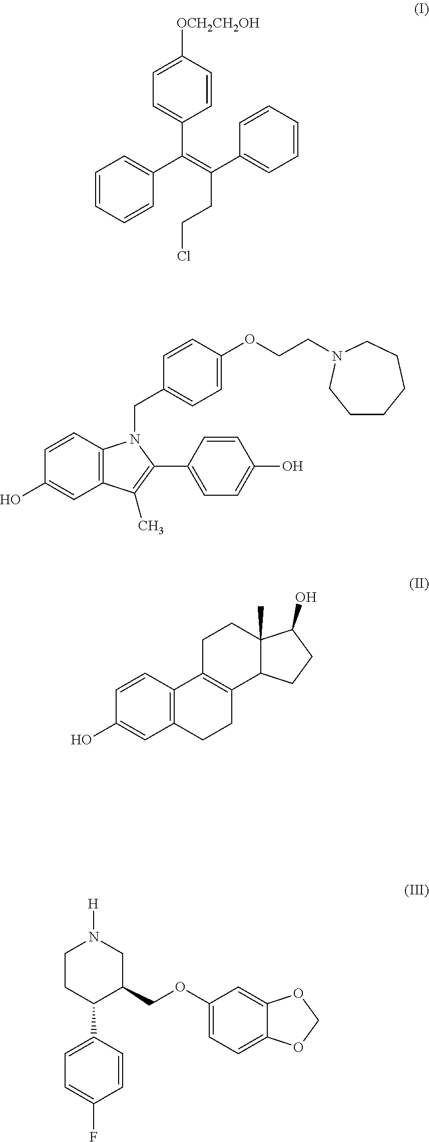

3. The method of claim 1, wherein the prescription medication is a non-estrogen therapy prescribed for the treatment of menopausal symptoms in women.

4. The method of any one of claims 1 to 3, wherein the subject is experiencing menopausal symptoms as a result of chemotherapy-induced menopause.

5. The method of any one of claims 1 to 3, wherein the subject is experiencing menopausal symptoms as a result of natural menopause.

6. The method of claims 4 and 5, wherein the natural product or combination of natural products is selected from the group consisting of black cohosh, a phytoestrogen, a cannabinoid, a terpene, an herbal product, and combinations thereof.

7. The method of claims 4 and 5, wherein the phytoestrogen is selected from the group consisting of an isoflavone, a lignan, a coumestan, and combinations thereof.

8. The method of claims 4 and 5, wherein the cannabinoid is selected from the group consisting of Δ⁸-THC, Δ⁹-THC, Δ⁸-THCA, Δ⁹-THCA, CBD, CBDA, CBC, CBN, CBG, CBGA, Δ⁸-THCV, Δ⁹-THCV, or their synthetic equivalents, and combinations thereof.

9. The method of claims 4 and 5, wherein the terpene is selected from the group consisting of α-bisabolol, trans-caryophyllene, p-cymene, guaiol, α-humulene, limonene, linalool, myrcene, cis-nerolidol, trans-nerolidol, α-pinene, β-pinene, terpinolene, or their synthetic equivalents, and combinations thereof.

10. The method of claim 5, wherein the herbal product is selected from the group consisting of Panax ginseng C. A. Meyer, Panax quinquefolium, Ginkgo biloba, curcumin, and any other natural product that may have benefits in the management of menopausal symptoms but cannot be classified as a phytoestrogen.

11. The method of any one of claims 1 to 10, wherein the effective amount of hormonal or non-hormonal prescription medication is the amount prescribed by the subject's physician.

12. The method of any one of claims 1 to 11, wherein the effective amount of natural product or combination of natural products is the amount that effectively alleviates chemotherapy-induced menopausal symptoms in the subject.

13. The method of any one of claims 1 to 11, wherein the effective amount of natural product or combination of natural products is the amount that effectively alleviates natural menopausal symptoms in the subject.

14. The method of claims 12 and 13, wherein the subject individually tailors the dose of natural product or combination of natural products to achieve satisfactory treatment of the subject's specific menopausal symptoms.

15. A kit comprising one or more natural products or combinations of natural products.

16. The kit of claim 15, further comprising a label with instructions for administering the one or more natural products or combinations of natural products in combination with the subject's prescription medication.

17. The kit of claims 15 and 16, wherein the natural product or combinations of natural products are selected from the group consisting of black cohosh, a phytoestrogen, a cannabinoid, a terpene, an herbal product, and combinations thereof.

18. The kit of claim 17, wherein the phytoestrogen is selected from the group consisting of an isoflavone, a lignan, a coumestan, and combinations thereof.

19. The kit of claim 17, wherein the cannabinoid is selected from the group consisting of Δ⁸-THC, Δ⁹-THC, Δ⁸-THCA, Δ⁹-THCA, CBD, CBDA, CBC, CBN, CBG, CBGA, Δ⁸-THCV, Δ⁹-THCV, or their synthetic equivalents, and combinations thereof.

20. The kit of claim 17, wherein the terpene is selected from the group consisting of α-bisabolol, trans-caryophyllene, p-cymene, guaiol, α-humulene, limonene, linalool, myrcene, cis-nerolidol, trans-nerolidol, α-pinene, β-pinene, terpinolene, or their synthetic equivalents, and combinations thereof.

21. The kit of claim 17, wherein the herbal product is selected from the group consisting of Panax ginseng C. A. Meyer, Panax quinquefolium, Ginkgo biloba, curcumin, and any other natural product that may have benefits in the management of menopausal symptoms but cannot be classified as a phytoestrogen.
